SEQUENCE LISTING (9 sequences)

SEQ ID NO: 1 - DNA, 20 bp, Oryza sativa
  source 1..20: mol_type = genomic DNA
gttggtctttgctcctgcag

SEQ ID NO: 2 - DNA, 23 bp, Oryza sativa
  source 1..23: mol_type = genomic DNA
agctgtaacaaaaggcccaaaag

SEQ ID NO: 3 - DNA, 23 bp, Oryza sativa
  source 1..23: mol_type = genomic DNA
accctccatcgaagccaaatatt

SEQ ID NO: 4 - DNA, 20 bp, Oryza sativa
  source 1..20: mol_type = genomic DNA
gcttctctacgtgcaaatgc

SEQ ID NO: 5 - DNA, 23 bp, Oryza sativa
  source 1..23: mol_type = genomic DNA
agggaaggagcaggagggttgtg

SEQ ID NO: 6 - DNA, 30 bp, Oryza sativa
  source 1..30: mol_type = genomic DNA
ggagtagtgatatgatttatttactgctac

SEQ ID NO: 7 - DNA, 20 bp, Oryza sativa
  source 1..20: mol_type = genomic DNA
ctccgtctgcgccatcgacc

SEQ ID NO: 8 - DNA, 18 bp, Oryza sativa
  source 1..18: mol_type = genomic DNA
tgctctccctcgacctcc

SEQ ID NO: 9 - DNA, 19 bp, Oryza sativa
  source 1..19: mol_type = genomic DNA
ttgtggaacgtgacgccat

(19) United States
(12) Patent Application Publication
(10) Pub. No.: US 2026/0167979 A1
(43) Pub. Date: Jun. 18, 2026

(54) METHOD FOR EDITING PLANT GENOME, PLANT BODY AND PLANT SEED GENOME-EDITED USING SAME, AND PRODUCTION METHOD THEREOF

(71) Applicants: Inplanta Innovations Inc., Kanagawa (JP); TOPPAN HOLDINGS INC., Tokyo (JP); NATIONAL INSTITUTE OF ADVANCED INDUSTRIAL SCIENCE AND TECHNOLOGY, Tokyo (JP)

(72) Inventors: Teruhiko TERAKAWA, Kanagawa (JP); Tsubasa YANO, Kanagawa (JP); Nobutaka MITSUDA, Ibaraki (JP); Akiyoshi NAKAMURA, Ibaraki (JP); Shigeo SUGANO, Ibaraki (JP); Seiichiro ITO, Tokyo (JP)

(73) Assignees: Inplanta Innovations Inc., Kanagawa (JP); TOPPAN HOLDINGS INC., Tokyo (JP); NATIONAL INSTITUTE OF ADVANCED INDUSTRIAL SCIENCE AND TECHNOLOGY, Tokyo (JP)

(21) Appl. No.: 18/711,320

(22) PCT Filed: Nov. 22, 2022
(86) PCT No.: PCT/JP2022/043204

§ 371 Date: May 17, 2024

(30) Foreign Application Priority Data
Nov. 22, 2021 (JP) 2021-189460

(51) Int. Cl. C12N 15/82; C12N 9/22; C12N 15/11

(52) U.S. Cl. CPC C12N 15/8213; C12N 9/226; C12N 15/11; C12N 15/8206

(57) ABSTRACT

Provided is a simple and efficient method of plant genome editing that does not involve the incorporation of a gene cassette for the expression system of a site-specific DNA modification protein. The method of the present invention comprises: (i) a step for mixing plant cells or a tissue containing plant cells together with a site-specific DNA modification protein and a needle-shaped inorganic compound in a liquid medium and making a disturbance to perforate the cells with the needle-shaped inorganic compound, thereby introducing the site-specific DNA modification protein into the cells; and (ii) a step for culturing the plant cells or the tissue containing the plant cells, into which the site-specific DNA modification protein has been introduced in step (i), thereby causing a DNA mutation specific to a target site in the genome of the plant cells.

### TECHNICAL FIELD

The present invention relates to a method of editing the genome of a plant, a genome-edited plant and a plant seed obtainable using the method, and a method for producing the same.

### BACKGROUND ART

A known genome editing technology employes a site-specific DNA-modifying protein. Examples of known site-specific DNA-modifying proteins include Cas proteins, zinc finger nucleases, and TAL effector nucleases (TALEN). An example of known Cas proteins is Cas9 nuclease. Cas9 nuclease is generally used as a complex with guide RNA. Introduction of a complex of Cas9 nuclease with guide RNA into a cell allows a target gene in a variety of plants and animals to be mutated. It is also known that two or more genes can be disrupted simultaneously by acting multiple guide RNAs simultaneously, and that various protein engineering modifications to Cas9 can be used to induce epigenetic dynamic changes.

Common methods of introducing foreign genes into plants that are currently in wide use are broadly classified into: indirect introduction methods such as Agrobacterium method, in which foreign genes are introduced indirectly via vectors into callus or tissue pieces in an in vitro culture system; and direct introduction methods such as implanter transformation method or protoplast-PEG method, in which foreign genes are introduced directly.

A representative example of the indirect introduction methods is the Agrobacterium method, which includes indirectly introducing a foreign gene into a plant cell by transforming the plant cell using Agrobacterium tumefaciens or other bacteria containing the foreign gene. However, this transformation method can only be applied to a limited species of plants and lacks versatility. In addition, when Cas9 nuclease is introduced into cells or tissues by the Agrobacterium method, the introduced Cas9 DNA is incorporated into the genome of the plant cell and constantly expressed in the plant cell, which increases the possibility of mutations occurring in DNA other than the target DNA (off-target). In addition, the final resulting plants are treated as transformants, thereby their use is limited. For this reason, it is more advantageous for the production of genome-edited plants to use the direct transfection methods, in which Cas9 DNA is not incorporated into the genome of the plant cells.

The implanter transformation method, an example of a direct introduction methods, includes introducing a gene directly into an exposed immature embryo or stem top using a particle gun (Patent Literature 1, Patent Literature 2, and Non-Patent Literature 1). However, the methods described in these literatures are challenged by low efficiency of gene transfer, as they are highly dependent on the manual skills of the experimenters. Accordingly, this method leaves room for improvement in terms of reproducibility.

The protoplast-PEG method, another example of the direct introduction methods, includes introducing a gene into a plant protoplast using polyethylene glycol (PEG) (Patent Literature 3). However, this method requires a lot of time and labor for the preparation of these objects, and also requires time-consuming and labor-intensive operations such as callusing and re-differentiation to obtain a genome-edited plant.

### CITATION LIST

### Patent Literature

[Patent Literature 1] JP2017-205104 A
[Patent Literature 2] JP2019-180373 A
[Patent Literature 3] JP2020-022455 A

### Non-Patent Literature

[Non-Patent Literature 1] Hamada et al. (2017) An in planta biolistic method for stable wheat transformation. Sci. Rep. 7 (1), 11443.

### SUMMARY OF INVENTION

### Problems to be Solved

An objective of the present invention s to provide a simple and efficient method for producing a genome-edited plant that does not involve the incorporation of an expression system gene cassette for a site-specific DNA-modifying protein.

### Means for Solving the Problems

The inventors have studied diligently to solve the aforementioned problems and, as a result, have arrived at the idea of mixing a plant cell or tissue with a site-specific DNA-modifying protein and an acicular inorganic compound (and optionally guide RNA) in liquid medium, or mixing a plant cell or tissue that constantly or inductively expresses a site-specific DNA-modifying protein with a guide RNA and an acicular inorganic compound in liquid medium, and then subjecting the mixture to disturbance, whereby the cell is perforated by the acicular inorganic compound and allows for the introduction of the site-specific DNA-modifying protein and/or the guide RNA therein, making it possible to obtain a genome-edited plant easily and efficiently. Based on these findings, the present inventors have completed the present invention.

The present invention includes the following aspects.

[Aspect 1] A method of editing the genome of a plant, comprising the steps of:
(i) mixing a plant cell or a tissue containing a plant cell with a site-specific DNA-modifying protein and an acicular inorganic compound in liquid medium and subjecting the mixture to disturbance to puncture the cell with the acicular inorganic compound and introduce the site-specific DNA-modifying protein into the cell; and
(ii) culturing the plant cell or the tissue containing the plant cell into which the site-specific DNA-modifying protein has been introduced at step (i) to generate a specific DNA mutation at a target site in the genome of the plant cell.

[Aspect 2] The method according to Aspect 1, wherein a guide RNA is co-mixed in the mixing at step (i).

[Aspect 3] A method of editing the genome of a plant, comprising the steps of:
(i) mixing a plant cell or a tissue containing a plant cell that constitutively or inductively expresses a site-specific DNA-modifying protein with a guide RNA and an acicular inorganic compound in liquid medium and subjecting the mixture to disturbance to puncture the plant cell with the acicular inorganic compound and cell and introduce the guide RNA into the cell, and
(ii) culturing the plant cell or the tissue containing the plant cell into which the guide RNA has been introduced to generate a specific DNA mutation at a target site in the genome of the plant cell.

[Aspect 4] The method according to any one of Aspects 1 to 3, wherein the site-specific DNA-modifying protein is selected from the group consisting of Cas protein, zinc finger motif, and TAL effector.

[Aspect 5]

The method according to Aspect 4, wherein the site-specific DNA-modifying protein forms a ribonucleoprotein (RNP) complex with the guide RNA.

[Aspect 6] The method according to any one of Aspects 1 to 5, wherein the concentration of the ribonucleoprotein complex is from 20 to 800 μmol.

[Aspect 7] The method according to any one of Aspects 1 to 6, wherein the maximum diameter of the plant cell or the tissue-contained plant cell is 1 mm or less.

[Aspect 8] The method according to any one of Aspects 1 to 7, wherein the disturbance at step (i) is carried out by centrifugation and/or ultrasonication.

[Aspect 9] The method according to Aspect 8, wherein the ultrasonication at step (i) is carried out by applying ultrasonic waves with a frequency of from 10 to 60 KHz and an intensity of from 0.1 to 1 W/cm² for 30 seconds to 2 minutes.

[Aspect 10] The method according to any one of Aspects 1 to 9, wherein a plasmid carrying a selection marker gene is co-mixed in the mixing at step (1).

[Aspect 11] The method according to any one of Aspects 1 to 10, wherein the culturing at step (ii) is carried out at a temperature of from 25 to 40° C. for 1 to 72 hours.

[Aspect 12] A method for producing a genome-edited plant, comprising the step of editing the genome of a plant cell step using the method according to any one of Aspects 1 to 11.

[Aspect 13] A method for producing a genome-edited plant seed, comprising the step of editing the genome of a plant cell step using the method according to any one of Aspects 1 to 11.

[Aspect 14] A genome-edited plant obtainable by the method according to Aspect 12.

[Aspect 15] A genome-edited plant seed obtainable by the method according to Aspect 13.

### Effects of the Invention

The present invention makes it possible to produce a genome-edited plant easily and efficiently without incorporation of an expression system gene cassette for a site-specific DNA-modifying protein.

### BRIEF DESCRIPTION OF DRAWINGS

FIG. 1 schematically illustrates overview of the genome editing operation of plant cells using an acicular inorganic compound (whisker).

FIG. 2 is a photograph of a rice plant whose OsPDS gene was genome-edited.

FIG. 3(a) is a photograph of a rice callus whose OsLCYβ gene was genome-edited, and FIG. 3(b) is a photograph of a rice callus which was not genome-edited.

FIG. 4 is a photograph of a rice plant whose OsLCYβ gene was genome-edited.

FIG. 5 illustrates an alignment of the DNA sequences of genome-edited OsLCYβ genes.

### EMBODIMENTS

The present invention will now be described in detail with reference to specific embodiments. However, the present invention is not limited to the following embodiments and can be implemented in any form without departing from the spirit of the present invention.

The Patent Literature documents (e.g., patent application publications and patent publications) and Non-Patent Literature documents cited herein are incorporated herein in their entirety for all purposes.

### *Plant Cells/Tissues to be Edited:

The target of the genome editing according to the present invention is plant cells and/or a tissue containing plant cells (e.g., a tissue of a part of a plant) (hereinafter collectively referred to as “plant cells/tissues” as appropriate).

Plants are not restricted as long as they can be a subject of genome editing with site-specific DNA-modifying proteins. Examples include rice, wheat, barley, corn, maize, oat, zoysia, sorghum, sugarcane, banana, etc. Examples of dicotyledonous plants include Arabidopsis, rape, cabbage, radish, soybean, azuki, kidney bean, pea, alfalfa, tomato, eggplant, potato, tobacco, pepper, cucumber, melon, watermelon, rose, strawberry, apple, rubber, cotton, lettuce, cyclamen, stevia, and torenia.

Examples of plant cells include de-differentiated cultured cells such as callus and suspension cells, as well as adventitious embryos. Examples of tissues containing plant cells include leaves, roots, stems, embryos, growth points, anthers, and pollens. Preferred among these include plant cells such as callus and suspension cells. The cultured cells used in the present invention may be any explant of plant origin, such as those derived from blastoderms, vegetative points, pollens, anthers, leaf blades, stems, petioles, and roots.

Tissues containing plant cells are not restricted as long as they are parts of plants. Examples include pollens, leaves, stems, roots, buds, flowers, fruits, and seeds. Plant parts may be in a state where they are not separated from the plant body, or they may be separated from the plant body.

Cultured cells to be used in the present invention can be obtained by culturing the explant mentioned above in callus-forming medium, such as medium based on MS medium (Murashige et al., “Physiologia Plantarum,” 1962, Vol. 15, pages 473 to 497), R2 medium (Ojima et al., “Plant and Cell Physiology,” 1973, Vol. 14, pages 1113 to 1121), or N6 medium (Chu et al., 1978, “In Proc. Symp. Plant Tissue Culture, Science Press Peking,” pages 43 to 50), containing inorganic salts and vitamins as essential components, 0.1 to 10 mg/L of 2,4-D (2,4-dichlorophenoxyacetic acid) as a plant hormone, and 10 to 60 g/L of sucrose as a carbon source, and 1 to 5 g/L of gelrite.

There is no particular limitation on the culture period since the placement of the explant on the callus-forming medium until the cultured cells for use in the present invention are obtained. However, for the purpose of obtaining a genome-edited plant, it is important that a plant can be regenerated from the cultured cells, i.e., that the plant cells possess the ability to regenerate a plant. The cultured cells to be used in the present invention may be suspension cells in liquid medium as long as the cultured cells possess the ability to regenerate a plant.

Plant cells or tissues that are the target of genome editing may constantly or inductively express a guide RNA or a site-specific DNA-modifying protein. In this case, it is not necessary to add the guide RNA or the site-specific DNA-modifying protein externally. Each of these embodiments will be described later.

### *Genome Editing:

The term “genome editing” as used herein refers to a technology to edit the genome in various cells by introducing a desired modification to a target site on the genome using, e.g., a site-specific DNA-modifying protein. Examples of the “modification” include, although are not limited to, disruption of a specific gene on the genome via cleavage, insertion or replacement of a DNA fragment at a target site on the genome, and modification of gene functions by introducing a point mutation at a high efficiency.

The term “target site” as used herein refers to a predetermined site in the genome of the plant cell to be subjected to genome editing. Such a target site can be appropriately selected according to the type of the site-specific DNA-modifying protein described below. For example, when Cas9, a Cas protein, is used as a site-specific DNA-modifying protein, the target site for genome editing may be a site on the genomic DNA consisting of a DNA strand (target strand) including a PAM (Proto-spacer Adjacent Motif) sequence and its 5′ flanking sequences of with a predetermined base length (which may preferably be, although is not limited to, within the range of 18 bases or more, especially 19 bases or more, and 25 bases or less, especially 22 bases or less, and may more preferably be 20 bases) and its complementary DNA strand (non-target strand). The number of base pairs upstream or downstream of the PAM sequence depends on the bacterial species from which Cas9 is derived, but most Cas9, including Cas9 derived from Streptococcus pyogenes, cleave three bases upstream of the PAM sequence.

The target site on the plant cell genome to be genome-edited is not particularly limited, but examples include a part or all of a gene in the plant cell genome that is desired to be modified or disrupted, or a region overlapping with or adjacent to such a gene. Examples of such genes include: genes related to primary metabolism (e.g., amino acids), genes related to secondary metabolism (e.g., flavonoids or polyphenols), genes related to sugar metabolism, genes related to lipid metabolism, genes related to production of useful substances (e.g., drugs, enzymes, pigments, or aromatic components), genes related to yields (e.g., numbers or sizes), genes related to flowering, genes related to disease insect resistance, genes related to tolerance to environmental stress (e.g., low temperature, high temperature, drought, salt, light damage, or ultraviolet light), etc.

### *Site-Specific DNA-Modifying Protein:

A site-specific DNA-modifying protein is used in the genome editing method of the present invention. According to an embodiment of the present invention, a site-specific DNA-modifying protein and a guide RNA are added and mixed with the target plant cells/tissues and the acicular inorganic compound. On the other hand, according to another embodiment, when the plant cells/tissues to be genome-edited expresses the guide RNA constitutively or inductively, it is not necessary to add the guide RNA externally, but only the site-specific DNA-modifying protein may be added and mixed with the plant cells/tissues and the acicular inorganic compound. According to still another embodiment, when the plant cells/tissues to be genome-edited expresses the site-specific DNA-modifying protein constitutively or inductively, it is not necessary to add the site-specific DNA-modifying protein externally, but only the guide RNA may be added and mixed with the plant cells/tissues and the acicular inorganic compound.

Examples of site-specific DNA-modifying proteins to be used for genome editing may include, although are not limited to, Cas protein, zinc finger nuclease (ZFN), and TAL effector nuclease (TALEN). Details of each group of proteins are described below.

The site-specific DNA-modifying protein may be selected from wild-type or known modified forms of the various proteins described below, or one or more mutations may be introduced to such wild-type or known modified proteins, as long as their activity is not impaired. Specifically, such a mutant site-specific DNA-modifying protein may preferably be derived from the amino acid sequence of a parent site-specific DNA-modifying protein via substitution, deletion, addition, or insertion of one or more amino acids, and may preferably have the same or a higher activity than the parent protein. In particular, a mutant site-specific DNA-modifying protein may preferably have an amino acid sequence having an identity of 90% or more, or 95% or more, or 97% or more, or 98% or more, or 99% or more to the amino acid sequence of the parent site-specific DNA-modifying protein. The “activity” in this context can be evaluated in vitro or in vivo according to known methods.

The site-specific DNA-modifying protein may be further linked to one or more other elements such as one or more peptides or proteins. For example, the site-specific DNA-modifying protein may have one or more nuclear localization sequences (NLS) at its N- and/or C-terminus.

### *Cas Protein:

The term “Cas protein” as used herein refers to a protein belonging to the Cas protein family, which constitutes an adaptive immune system for bacteria and archaea to exhibit acquired resistance to invading foreign nucleic acids. The Cas protein is a target specific endonuclease that recognizes a PAM sequence and cleaves a double-stranded DNA upstream or downstream thereof. The Cas protein is an RNA-guided nuclease (RGN) and constitutes a CRISPR (clustered regularly interspaced short palindromic repeat)/Cas system together with a guide RNA (gRNA). When the CRISPR/Cas system is introduced or constructed in a target cell, the guide RNA binds to the target site in the genome, whereby the Cas protein can be recruited to the binding site and cleave DNA at the target site. The combination of the Cas protein and the guide RNA may be either a naturally occurring combination or a combination that does not exist in nature.

Examples of Cas protein families include Cas1, Cas1B, Cas2, Cas3, Cas4, Cas5, Cas6, Cas7, Cas8, Cas9, Cas10, Cas12, Csy1, Csy2, Csy3, Cse1, Cse2, Csc1, Csc2, Csa5, Csn2, Csm2, Csm3, Csm4, Csm5, Csm6, Cmr1, Cmr3, Cmr4, Cmr5, Cmr6, Csb1, Csb2, Csb3, Csx17, Csx14, Csx10, Csx16, CsaX, Csx3, Csx1, Csx15, Csf1, Csf2, Csf3, and Csf4, and homologs or modified variants thereof. Preferred among these as Cas proteins to be used in the present invention include Cas9, Cas12, and homologs or modified variants thereof, especially preferably Cas9.

Examples of bacterial species from which Cas proteins are derived may include, although are not limited to, Streptococcus pyogenes (S. pyogenes), Streptococcus aureus, Francisella novicida, Streptococcus thermophilus, Nocardiopsis dassombiae, Streptomyces pristinaespiralis, Streptomyces viridochromogenes, Streptosporangium roseum, Alicyclobacillus acidocaldarius, Bacillus pseudomycoides, Bacillus selenitireducens, Exiguobacterium sibiricum, Lactobacillus delbrueckii, Lactobacillus salivarius, Microcilla marina, Burkholderia bacteria, Polaromonas naphthalenivorans, Polaromonas spp., Crocosphaera watsonii, Cyanoseis spp., Microcystis aeruginosa, Synechococcus spp., Acetohalobium arabicum, Ammonifex degensii, Caldicellulosiruptor becscii, Candidatus Desulforudis, Clostridium botulinum, Clostridium difficile, Finegoldia magna, Natranaerobius thermophilus, Perotomaculum thermopropionicum, Acidithiobacillus caldus, Acidithiobacillus ferrooxidans, Allochromatium vinosum, Marinobacter spp., Nitrosococcus halophilus, Nitrosococcus watsonii, Pseudoalteromonas haloplanktis, Ktedonobacter racemifer, Methanohalobium evestigatum, Anabaena variabilis, Nodularia spumigena, Nostoc spp., Arthrospira maxima, Arthrospira platensis, Arthrospira spp., Lyngbya spp., Microcoleus chthonoplastes, Oscillatoria spp., Petrotoga mobilis, Thermosipho africanus, and Acaryochloris marina. Preferred among these as bacteria from which Cas9 is derived may include Streptococcus pyogenes.

For details of CRISPR/Cas systems and Cas proteins, refer to, e.g., WO2014/093595A, WO2014/093635A, WO2015/089473A, WO2019/060469A, WO2019/217336A, WO2019/217336A, WO2013/176772A, and WO2013/142578A.

Technologies derived from the CRISPR/Cas system include: the CRISPR/Cpf1 system, which is based on a Cpf1 (Cas12a) protein, a DNA endonuclease involved in the Class 2V CRISPR/Cas system; and the CRISPR/dCas9-BE system, which is based on a base editor (BE), a fusion of a dCas9 (a Cas9 whose nuclease activity has been inactivated) with deaminase (deamination enzyme). Proteins constituting these systems may also be included in the “Cas proteins” according to the present invention.

### *Zinc Finger Nuclease (ZEN):

A zinc finger nuclease (ZFN) is a fusion protein containing several zinc finger motifs that recognize specific bases and a FokI nuclease fused therewith. The zinc finger nuclease typically contains a zinc finger domain, which binds to a specific target site in a nucleic acid molecule, and a nucleic acid cleavage domain, which cleaves the nucleic acid molecule within or proximal to the target site bound by the binding domain. For details, see, e.g., Curtin et al, Plant Physiol. (2011), 156 [2]: 466-73, et al.

### *TAL Effector Nuclease (TALEN):

A TALEN is a fusion protein containing a Transcription Activator Like (TAL) effector and a FokI nuclease fused therewith. A TAL effector nuclease (TALEN) is an artificial nuclease containing a transcription activator-like effector DNA-binding domain (TALE) in a DNA cleavage domain (e.g., FokI domain), and is an effector protein containing a DNA-binding domain including a highly conserved sequence with 33-34 amino acids. For details, see, e.g., Li et al. Nat. Biotechnol. (2012), 30 [5]: 390-2 and WO2020/045281A.

### *Guide RNA:

The guide RNA is an RNA that functions to direct (guide) the site-specific DNA-modifying protein to the target site on the genome. The site-specific DNA-modifying protein usually binds to the guide RNA to form a ribonucleoprotein or ribonucleoprotein (RNP). In this context, the guide RNA targets the target site on the genome, and the site-specific DNA-modifying protein cleaves the DNA at the target site on the genome, thereby altering or modifying the DNA sequence at the target site on the genome.

The structure of the guide RNA may typically be selected based on the type of the site-specific DNA-modifying protein. For example, when a Cas protein is used as the site-specific DNA-modifying protein, the guide RNA usually contains a crRNA (CRISPR RNA) sequence, which is involved in the activity of the CRISPR/Cas system, and a tracrRNA (trans-activating crRNA) sequence, which binds to a target site on the genome. In this case, the guide RNA may be a single-stranded RNA (sgRNA) containing a crRNA sequence and a tracrRNA sequence, or an RNA complex consisting of an RNA containing a crRNA sequence and an RNA containing a tracrRNA sequence complementarily bound to each other. The tracrRNA sequence may typically be, although is not limited to, an RNA consisting of a sequence of about 50 to 100 bases in length that can form multiple stem loops. The appropriate RNA sequence can be selected and used according to the target site on the genome to be edited and the type of the Cas protein to be used together.

The length of the guide RNA may preferably be, although are not limited to, 15 nucleotides or more, or 18 nucleotides or more, and 30 nucleotides or less, or 25 nucleotides or less, or 22 nucleotides or less. Among others, the length of the guide RNA may preferably be about 20 nucleotides.

As mentioned above, according to an embodiment of the present invention, it is preferable to add and mix the guide RNA in addition to the site-specific DNA-modifying protein with the plant cells/tissues and the acicular inorganic compound. On the other hand, according to another embodiment, when the plant cells/tissues to be genome-edited expresses the guide RNA constantly or inductively, it is not necessary to add the guide RNA externally, so the use of guide RNAs is not required. According to still another embodiment, when the plant cells/tissues to be genome-edited expresses the site-specific DNA-modifying protein constantly or inductively, it is not necessary to add the site-specific DNA-modifying protein externally, but only the guide RNA may be added and mixed with the plant cells/tissues and the acicular inorganic compound.

### *Ribonucleoprotein (RNP):

The site-specific DNA-modifying protein may typically bind to the guide RNA or other RNAs such as transition RNA (tRNA) and messenger RNA (mRNA) to form a ribonucleoprotein (RNP). This allows an appropriate genome editing system to be established in the plant cell depending on the type of the site-specific DNA-modifying protein (e.g., when a Cas protein is used as the site-specific DNA-modifying protein, the CRISPR/Cas system is established) to make the desired modification to the target site on the plant cell genome.

As mentioned above, according the embodiment of the present invention where the site-specific DNA-modifying protein and the guide RNA are added and mixed with the plant cells/tissues and the acicular inorganic compound, the site-specific DNA-modifying protein molecules bind to the guide RNA molecules to form ribonucleoprotein (RNP) molecules, and the formed RNP molecules are incorporated into the plant cell through pores perforated in the cell wall by the acicular inorganic compound molecules (in this case, of course, unbound molecules of the site-specific DNA-modifying protein and/or the guide RNA may also be incorporated into the plant cell together).

According to the embodiment of the present invention where the plant cells/tissues expressing the guide RNA constitutively or inductively is used and mixed with the acicular inorganic compound and the site-specific DNA-modifying protein, molecules of the site-specific DNA-modifying protein are incorporated into the plant cell through pores perforated in the cell wall by the acicular inorganic compound molecules, and bind to molecules of the guide RNA expressed in the plant cell constantly or inductively, thereby forming ribonucleoprotein (RNP) molecules.

According to the embodiment of the present invention where the plant cells/tissues expressing the site-specific DNA-modifying protein constitutively or inductively is used and mixed with the acicular inorganic compound and the guide RNA, molecules of the guide RNA are incorporated into the plant cell through pores perforated in the cell wall by the acicular inorganic compound molecules, and bind to molecules of the site-specific DNA-modifying protein expressed in the plant cell constantly or inductively, thereby forming ribonucleoprotein (RNP) molecules.

### *Acicular Inorganic Compound:

The term “acicular inorganic compound” as used herein refers to a single-crystal inorganic compound with a fine acicular structure. According to the present invention, when the plant cells/tissues is mixed with the acicular inorganic compound in a liquid medium and then subjected to disturbance, the acicular inorganic compound pierces the plant cell and perforates its cell wall, through which the site-specific DNA-modifying protein and/or the guide RNA and/or the ribonucleoprotein (RNP) may be introduced into the plant cell.

There are no restrictions on the type of acicular inorganic compound as long as it is capable of piercing plant cells and perforating their cell walls when disturbed. One example of such an acicular inorganic compound is an acicular inorganic compound called whisker. Whisker is an acicular single crystal known as an industrial material. For details, see, e.g., “Whisker: Introduction to Ultra High Strength Single Crystals,” by Yoshinori Fujiki and Mamoru Mitomo, published by Sangyo Tosbo Publishing Co., Ltd., 1993,

When a whisker is used as the acicular inorganic compound, specific examples of the material for the whisker include, although are not limited to, potassium titanate, calcium carbonate, aluminum borate, silicon nitride, zinc oxide, basic magnesium sulfate, magnesia, magnesium borate, titanium diboride, carbon graphite, calcium sulfate, sapphire, and silicon carbide, preferred among which are potassium titanate, calcium carbonate, and aluminum borate.

When a whisker is used as the acicular inorganic compound, the size of the whisker is not particularly limited, but its diameter may preferably be 0.01 μm or more, or 0.5 μm or more, and 10 μm or less, or 1 μm or less, and its length may preferably be 1 μm or more, or 3 μm or more, and 100 μm or less, or 40 μm or less.

When a whisker is used as the acicular inorganic compound, it is possible to use a whisker as it is, but it is preferable to use a whisker that has been surface-treated with a surface treatment agent, more preferably a whisker that has been surface-treated such that its surface has basic functional groups. The use of such surface-treated whiskers allows for more efficient perforation of the plant cell and increases the rate of introduction of the site-specific DNA-modifying protein and/or the guide RNA and/or the ribonucleoprotein (RNP). The basic functional groups may include those derived from primary to quaternary amines and divalent metal complexes, preferred among which are amino groups.

When a surface-treated whisker is used as the acicular inorganic compound, the surface treatment agent may be any compounds as long as they can covalently bond to whisker surfaces, examples including silane coupling agents. Preferred among these include silane coupling agents having basic groups. Specific examples of such silane coupling agents include, although are not limited to, basic silane coupling agents such as 3-(2-aminoethoxylaminopropyl)-trimethoxysilane and 3-aminopropyl-triethoxysilane.

### *Selection Marker Gene:

A plasmid containing a selection marker gene may be used together with the RNP and the acicular inorganic compound. In this case, examples of plasmid expression vectors include, although are not limited to, pUC vectors (e.g., pUC18, pUC19, and pUC9) and pBI vectors (e.g., pBI121, pBI101, pBI221, pBI2113, and pBI101.2). It may constitute an expression vector for a drug resistance gene.

Examples of drug resistance genes include medicament resistance gene (e.g., tetracycline resistance gene, ampicillin resistance gene, kanamycin resistance gene, hygromycin resistance gene, spectinomycin resistance gene, chloramphenicol resistance gene, and neomycin resistance gene), herbicide resistance genes (e.g., viaraphos resistance gene, glyphosate resistance gene (EPSPS), and sulfonylurea resistance gene (ALS)), and fluorescent or luminescent reporter genes (e.g., luciferase, β-galactosidase, β-glucuronidase (GUS), and green fluorescein protein (GFP)).

### *Plant Genome Editing Method:

The genome editing method according to an embodiment of the present invention includes at least the steps of:
(i) mixing a plant cells/tissues with a site-specific DNA-modifying protein, a guide RNA and an acicular inorganic compound in liquid medium and subjecting the mixture to disturbance to puncture the cell with the acicular inorganic compound and introduce the site-specific DNA-modifying protein into the cell; and
(ii) culturing the plant cell or the tissue containing the plant cell into which the site-specific DNA-modifying protein has been introduced at step (i) to generate a specific DNA mutation at a target site in the genome of the plant cell.

The details of each of the steps are explained below.

### *Step (i): Mixing

This step is to mix a plant cells/tissues with a site-specific DNA-modifying protein, a guide RNA, and an acicular inorganic compound in liquid medium and subject the mixture to disturbance.

Any liquid medium can be used as the liquid medium for this step. Examples include distilled water, buffer solution, isotonic solution, and culture medium for tissue culture. Examples of buffer solutions include phosphate buffer, Tris buffer, and MES buffer. Examples of isotonic solutions include liquid media prepared by adding inorganic salts such as KCl, NaCl, CaCl₂), and MgCl₂ to distilled water and adjusting their concentrations to within the range of, e.g., 0.01 M or more, or 0.5 M or more, and 7 M or less, or 2 M or less. Examples of tissue culture media include MS medium, Gamborg's B5 medium, R2 medium, White's medium, Niche-Niche medium, and N6 medium. The pH of the liquid medium is not restricted, but may preferably be within the range of pH 6 or more and pH 8 or less, more preferably pH 7.5.

In this step, the amount of plant cells and tissues in the liquid medium is not particularly limited, but may be adjusted so that each mL of the liquid medium contains 1×10³ cells or more, or 1×10⁴ cells or more, or 1×10⁵ cells or more, and 1×10⁸ cells or less, or 1×10⁷ cells or less, or 1×10⁶ cells or less.

In this step, the concentration of the acicular inorganic compounds in the liquid medium is not particularly limited, but may be adjusted appropriately according to the type of the acicular inorganic compound and the type and amount of the plant cells/tissues. For example, it is preferable to adjust the concentration of the acicular inorganic compound per mL of the packed cell volume (hereinafter referred to as “PCV”) of plant cells to within the range of 1 mg or more, or 4 mg or more, and 100 mg or less, or 40 mg or less.

In this step, the amount of the site-specific DNA-modifying protein and the guide RNA, if used, in the liquid medium is not limited, but it may preferably be adjusted so that a predetermined amount of ribonucleoprotein (RNP) is formed. Specifically, it is preferable to adjust the amount(s) of the site-specific DNA-modifying protein and/or the guide RNA such that the amount of the ribonucleoprotein (RNP) formed in the liquid medium is within the range of 20 μmol or more, or 100 μmol or more, and 800 μmol or less, or 600 μmol or less.

In this step, the plant cells/tissues, the site-specific DNA-modifying protein, the guide RNA, and the acicular inorganic compound are mixed in the liquid medium in the same container. Containers are not limited as long as they can handle plant cells/tissues in a sterile manner. Examples include microtubes, centrifuge tubes, glass test tubes, polypropylene test tubes, petri dishes, and flasks. Usually, the container is shaken and agitated so that the plant cells/tissues, the site-specific DNA-modifying protein, the guide RNA, and the acicular inorganic compound placed in the container are uniformly mixed and dispersed in the liquid medium, resulting in a mixture of the plant cells/tissues, the acicular inorganic compound, and the ribonucleoprotein (RNP) (formed by the binding of the site-specific DNA-modifying protein and the guide RNA) dispersed in liquid medium.

As explained above, according to the embodiment of the genome editing method of the present invention where the plant cells/tissues constitutively or inductively express the site-specific DNA-modifying protein or the guide RNA, it is not necessary to add the site-specific DNA-modifying protein or the guide RNA externally. Specifically, when the plant cells/tissues constitutively or inductively express the site-specific DNA-modifying protein, only the acicular inorganic compound and the guide RNA should be added to the plant cells/tissues in the same container and mixed in the liquid medium mixing. On the other hand, when the plant cells/tissues constitutively or inductively express the guide RNA, only the acicular inorganic compound and the site-specific DNA-modifying protein should be added to the plant cells/tissues in the same container and mixed in the liquid medium mixing.

### *Step (i): Disturbance

In this step, the mixture is then subjected to disturbance. The method of applying disturbance is not limited, and any method can be used. Examples include any one treatment selected from centrifugation, ultrasonication, and vortex mixing, etc., as well as combinations of two or more of these treatments.

According to the genome editing method of the present invention, the plant cells/tissues is subjected to disturbance in this step in the presence of the acicular inorganic compound to thereby puncture the cell wall. It is assumed that this will allow the formation of sufficient pores in the cell wall to allow the site-specific DNA-modifying protein and/or the guide RNA and/or the ribonucleoprotein (RNP) to enter the cell without causing significant damage to the cell. It is also assumed that this makes it possible to reduce off-targets compared to conventional genome editing methods, as well as to simultaneously edit multiple target sites and improve the efficiency of genome editing of large numbers of cells.

In this step, it is preferable to carry out centrifugation and ultrasonication in this order. According to this embodiment, the acicular inorganic compound is attached to the plant cell via centrifugation and then made to vibrate via ultrasonication, which enables more efficient perforation of the cell wall of the plant cell by the acicular inorganic compound.

When centrifugation is performed, the conditions are not limited, but examples are as follows. The centrifugal acceleration may preferably be 3,000×g or more, or 10,000×g or more, and 50,000×g or less, or 30,000×g or less. The centrifugation time may preferably be at 10 seconds or more, or 5 minutes or more, and 20 minutes or less, or 10 minutes or less. Although centrifugation may be performed at least once, it is preferable to repeat the same centrifugation process two or more times or three or more times to increase the amount of the acicular inorganic compound attached to the plant cells. The maximum number of repetitions is not limited, but may usually be 10 times or less.

When ultrasonication is performed, the conditions are not limited, but may preferably be gentler than the ultrasonication conditions used in the various conventional techniques described above. Examples are as follows. The frequency of ultrasonic waves may preferably be 1 kHz or more, or 10 KHz or more, and usually 1 MHz or less, or 60 kHz or less. The irradiation time of ultrasonic waves may preferably be 0.2 seconds or more, or 30 seconds or more, and 20 minutes or less, or 2 minutes or less. The intensity of ultrasonic waves may preferably be 0.01 W/cm² or more, or 0.1 W/cm² or more, and 10 W/cm² or less, or 1 W/cm² or less.

### *Step (ii): Culture

After the mixing and disturbance treatment of step (i), the resulting mixture may preferably be allowed to stand still. This allows the site-specific DNA-modifying protein and/or the guide RNA and/or the ribonucleoprotein (RNP) to fully penetrate and diffuse into the cell through the pores in the plant cell wall perforated by the acicular inorganic compound. The conditions for the standstill treatment are not restricted, but examples are as follows. The temperature during the standstill treatment may preferably be 0° C. or more, 4° C. or more, and 40° C. or less, or 35° C. or less. The time of the standstill treatment may preferably be 1 minute or more, or 5 minutes or more, and 3 hours or less, or 1 hour or less.

After the mixing and disturbance treatment of step (i), preferably after the standstill treatment mentioned above, the mixture may be cultured as is, but may preferably be washed to remove the acicular inorganic compound from the mixture before being cultured. Although there are no particular restrictions on the washing solution to be used for washing, it is usually preferable to use distilled water, isotonic solution, buffer solution, culture medium, etc., as in the examples of liquid media described above, with isotonic solution or culture medium being especially preferable. The method of washing is not limited, but an example includes removing the liquid phase from the mixture, and then carrying out the washing operation of adding washing solution to the container and mixing it repeatedly several times. Removal of the liquid phase may be carried out by using an operation such as peroxidation, whereby the solid phase containing plant cells and tissues can be separated from the liquid phase more efficiently.

The plant cells/tissues thus obtained may be subsequently subjected to culture. This will result in the expression of a genome editing system constructed with the site-specific DNA-modifying protein and the guide RNA in the plant cell, and genome editing will be performed on the target site on the plant cell genome. The conditions of culture are not restricted, but examples are as follows.

The type of culture medium is not limited, and any medium suitable for culturing plant cells/tissues can be used. The medium may be a liquid medium or a solid medium. Examples of liquid media include MS medium, Gamborg's B5 medium, R2 medium, White's medium, Niche-Niche medium, and N6 medium, which were also mentioned above as examples of liquid media. Solid culture media include the aforementioned liquid culture media solidified with agar or the like. Various additives may be added to these media as needed. Examples of additives include plant hormones and carbon sources. Examples of plant hormones include auxins such as 2,4-D, naphthyl acetic acid, and indole acetic acid, and cytokinins such as benzyladenine and kinetin. Carbon sources include sucrose and glucose. The type and combination of these media and additives may be selected according to the target plant species.

The temperature during culture is not particularly restricted, but may usually be 15° C. or more, or 20° C. or more, and 40° C. or less, or 35° C. or less.

The incubation time is also not limited, but may usually be 1 hour or more, 3 hours or more, 12 hours or more, or 24 hours or more. The upper limit is also not limited, but may be 14 days or less, or 7 days or less, or 120 hours or less, or 72 hours or less.

### *Other Steps

The above procedure results in a dividing cell mass (callus) containing genome-edited cells. However, such a cell mass contains a mixture of genome-edited cells and non-genome-edited cells. In order to efficiently obtain genome-edited cells and plant, it is desirable to carry out an operation to select and separate genome-edited cells from dividing cells.

Such an operation may be carried out by mixing a plasmid carrying a drug resistance gene as a selection marker (selection marker plasmid) in the liquid medium at the time of mixing in step (i) above. This makes it possible to efficiently select genome-edited cells by using the drug resistance effect to select dividing cells in which the site-specific DNA-modifying protein and/or the guide RNA and/or the ribonucleoprotein (RNP) have been introduced with the selection marker plasmid.

There are no particular restrictions on the drug resistance genes that serve as selection markers, and any conventionally known drug resistance gene can be selected and used, depending on the type of plant and the type of modification introduced by genome editing. Examples include known antibiotic resistance genes such as hygromycin and kanamycin. The method of constructing the selection marker plasmid is also not limited, and various known methods can be selected and used as appropriate.

After the mixing and disturbance in step (i) are carried out in the presence of the selection marker plasmid, followed by culture in step (ii), the resulting meristematic cell mass may be placed or suspended in solid or liquid selection medium and cultured. The selection medium can be the aforementioned medium for plant tissue culture plus an appropriate agent, such as hygromycin, kanamycin, etc., according to the resistance substance of the selection marker. The concentration of the agent is not limited, and may be selected according to the type of plant and the type of agent. To give an example, the concentration may be usually 1 mg/L or more and 300 mg/L or less, or 25 mg/L or more and 50 mg/L or less. The incubation time is also not limited, but may be usually 1 day or more, or 3 days or more, and 60 days or less, or 40 days or less.

### *Generation of a Genome-Edited Plant and Plant Seeds

The genome editing method of the present invention described above produces genome-edited plant cells/tissues, which can be cultured further to produce a genome-edited plant or plant seeds.

Specifically, genome-edited plant cells/tissues obtained as described above may be placed on known medium for plant regeneration and cultured to obtain a genome-edited plant. The conditions during culture are not limited and may be selected according to the type of plant and the type of modification to be introduced by genome editing. For example, the temperature of culture may be usually 15° C. or more, or 20° C. or more, and 30° C. or less, or 28° C. or less. The light irradiation during incubation may usually be 500 lux or more, or 800 lux or more, and 2,000 lux or less, or 1,000 lux or less. The incubation period may be 20 days or more, or 30 days or more, and 60 days or less, or 40 days or less.

The resulting genome-edited plant can be fertilized and bear fruit, and its seeds can be collected as genome-edited plant seeds.

### EXAMPLES

The present invention will now be described in more detail with reference to Examples. It should be noted that the present invention is not limited to the following Examples and can be implemented in any form without departing from the spirit of the present invention.

### [Example 1] Genome Editing Operation on Plant Cells

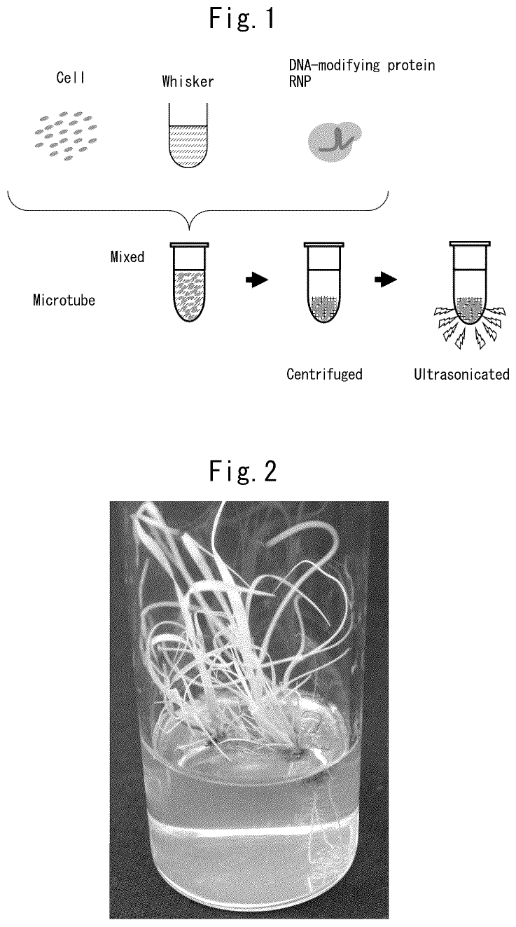

FIG. 1 shows a schematic overview of genome editing operations on plant cells using an acicular inorganic compound (whisker). Specifically, the following procedure was used.

### (1) Preparation of Test Plant Cells

Rice (scientific name: Oryza sativa (Os); cultivar: Nipponbare) was sterilized by threshing the hulls of ripe seeds and immersing them in a 70% ethanol solution for 10 seconds, then in a 1% effective chlorine sodium hypochlorite solution for 6 minutes, followed by washing with sterile water. 30 g/L sucrose, 2,4-D 2 mg/L, and 0.3 g/L of gelite (pH 5.8) were added to the inorganic component composition of MS medium, and the medium was solidified in a petri dish 90 mm in diameter to prepare a solid medium. 9 seeds obtained above were placed in one petri dish on this solid medium and incubated at 28° C. for 14 days in the light (2,000 lux, 16 hours of illumination per day) to thereby produce calluses. Liquid culture medium (pH 5.8) was prepared by adding 30 g/L sucrose, 2 mg/L 2-4-D, and 2 g/L casamino acid to the inorganic component of the R2 medium and placing 50 mL in a 100-mL triangular flask and sterilizing it by autoclaving (this liquid culture medium is hereinafter referred to as “R2D2 medium”).

The calluses obtained above were cut from the endosperm and transferred to the R2D2 medium such that each flask contains 10 calluses, which were then cultured at a temperature of 28° C. with shaking using a rotary shaker (100 rpm/min) in the light (2,000 lux, 16 hours illumination per day), thereby producing suspension-cultured cells. The suspension-cultured cells were passaged every 7 days by transferring 3 mL of PCV into fresh R2D2 medium. After 28 days of passaging culture, the resulting calluses were filtered via a stainless-steel mesh sieve with 1 mm openings, whereby calluses with a size of 1 mm or less were obtained at a PCV of 3 mL per petri dish. The resulting rice calluses of 1 mm or less were washed three times with R2D2 medium and used for testing.

### (2) Preparation of an Acicular Inorganic Compound

One gram of potassium titanate whisker (product “LS20”; Titan Kogyo, Ltd.) was placed in a recovery flask with a 500-mL volume, into which 100 mL of toluene and 1 g of 3-(2-aminoethoxylaminopropyl)-3-(2-aminoethoxylamino)-trimethoxysilane (coupling agent) were added and dissolved. The toluene in the flask was then heated to 120° C. with stirring, whereby toluene was distilled off a slurry was obtained. After the reaction was completed, the slurry was washed with 90% methanol to remove excess coupling agent. The remaining methanol used for washing was completely removed using a rotary evaporator to obtain surface basic whiskers. 5 mg of the surface basic whisker thus obtained was placed in a 1.5-mL tube (Eppendorf), combined with 0.5 mL of ethanol, and left to stand overnight. The ethanol was completely evaporated to obtain sterilized whiskers. The tube containing the whiskers was filled with 1 mL of sterile water, stirred well, centrifuged at 3000 rpm/min for 5 minutes, and the supernatant was discarded to wash the whiskers. After this washing operation was repeated three times, 0.5 mL of R2D2 medium was added to the tube to obtain a whisker suspension.

### (3) Preparation of a RNP Complex

RNP was prepared by in vitro transcription using the Guide-it™ sgRNA In Vitro Transcription Kit (Takara Bio Inc.). Specifically, a RNP solution was prepared by distilling sgRNA and Cas9 in RNase-free gel filtration buffer (20 mM Tris-HCl pH 7.5, 150 mM NaCl, 10% (v/v) glycerol, 1 mM MgCl₂), and left to stand at 25° C. for 10 minutes to form a complex. 2 μL of 1 mg/mL polyomithine solution was added per 98 μL of the RNP solution and allowed to react for 10 minutes. The treated solution was filter-sterilized and used as the RNP solution for transfection.

### (4) Introduction of RNP Complex

Calluses with a size of 1 mm or less obtained in (1) above in a PCV of 250 μL was added to a 1.5-mL tube containing the whisker suspension obtained in (2) above. After agitation, the calluses and the whisker were centrifuged at 1,000 rpm/min for 10 seconds to precipitate the calluses and the whisker, and the supernatant was discarded to obtain a mixture of the calluses and the whisker. The RNP solution obtained in (3) above was added to the tube containing the mixture of the calluses and the whisker above.

Next, the tube containing this mixture was centrifuged at 15,000×g for 5 minutes, and then shaken again. This operation of centrifugal separation and shaking was repeated three times. The tube containing the mixture thus treated was then placed in a bathtub with an ultrasonic generator (bathtub type: water was used as the medium) so that the tube was fully submerged. The tube was irradiated with ultrasonic waves at a frequency of 40 kHz

### [Example 2] Genome Editing of the OsPDS Gene in Cultured Rice Cells

### (1) Preparation of Test Plant Cells

The procedure described in Example 1 (1) was followed.

### (2) Preparation of an Acicular Inorganic Compound

The procedure described in Example 1 (2) was followed.

### (3) Preparation of a RNP Complex

The target sequence of the rice (Os) phytoene desaturase (PDS) gene is indicated as SEQ ID NO:1. The procedure described in Example 1 (3) was followed, except that the concentration of RNP solution was adjusted to 100 μmol.

*Target sequence of the OsPDS gene (sequence of the region complementary to the sgRNA sequence)

OsPDS: GTTGGTCTTTGCTCCTGCAG (SEQ ID NO: 1)

### (4) Introduction of RNP Complex

The procedure described in Example 1 (4) was followed.

### (5) Culturing of Dividing Cells and DNA Sampling

The RNP-transfected calluses cultured in (4) were incubated at 35° C. for 1 hour and 24 hours and then sampled. Sampled calluses were quenched at −80° C. and mashed using a masher. The mashed sample was combined with 300 μL of DNA extraction solution (100 mM Tris (pH 8.0), 50 mM EDTA (pH 8.0), 500 mM NaCl), and mixed uniformly with a vortex mixer. The resulting mixture was then combined with 15 μL of 20% SDS solution and incubated at 65° C. for 10 minutes. After the incubation, the mixture was then combined with 90 μL of 5M potassium acetate, mixed gently, and centrifuged at 15,000×g for 5 minutes. The supernatant was transferred to a new 1.5-mL tube, into which an equal volume of isopropanol was added, and the tube was inverted and mixed. After discarding the supernatant, 500 μL of 70% ethanol was added, and centrifuged at 15,000×g for 2 minutes, and the supernatant was again discarded. The precipitate was air-dried for 10 minutes and then dissolved in 100 μL of sterile MilliQ water. The extracted DNA sample was used as a template for PCR amplification of the target site. PCR was performed on the extracted DNA sample using primers (SEQ ID NO: 2 and 3). KOD one was used as the PCR enzyme. An amplification cycle of 98° C.: 10 seconds, 55° C.: seconds, and 68° C.: 1 second was repeated for 35 cycles.

*Primer sequences
CsPDS_Fw:
(SEQ ID NO: 2)
AGCTGTAACAAAAGGCCCAAAAG
CsPDS_Rv:
(SEQ ID NO: 3)
ACCCTCCATCGAAGCCAAATATT

### (6) Evaluation of Genome Editing Efficiency

In the DNA fragments of unedited cells, the PCR product was completely cleaved by the restriction enzyme Pstl, whereas in the DNA fragments of mutagenized cells, the PCR product left a residue. DNA was extracted from the remaining band, purified, and subjected to sequencing analysis, and those with mutations in the target gene sequence were determined to be target-gene mutant cells. As a result, it was confirmed that the mutation was introduced into the OsPDS target gene in the cells.

TABLE 1
Genome editing of the OsPDS gene

| RNP | Culture | Culture | Editing |
|---|---|---|---|
| concentration | time | temperature | rate |
| (pmol) | (hours) | (° C.) | (%) |
| 100 | 1 | 25 | 0.07 |
| 100 | 1 | 35 | 0.10 |
| 100 | 24 | 35 | 0.18 |

### [Example 3] Genome Editing of the OsLCY: 5 Gene in Cultured Rice Cells

### (1) Preparation of Test Plant Cells

The procedure described in Example 1 (1) was followed.

### (2) Preparation of an Acicular Inorganic Compound

The procedure described in Example 1 (2) was followed.

### (3) Preparation of a RNP Complex

The target sequence of the rice (Os) lycopene εcyclase (lycopene ε-cyclase: LCYε/LCYe5) gene is indicated as SEQ ID NO:4. The procedure described in Example 1 (3) was followed, except that the concentration of RNP solution was adjusted to 100 to 600 μmol.

*Target sequence of the OsLCYε gene (sgRNA sequence)

OsLCYε: GCTTCTCTACGTGCAAATGC (SEQ ID NO:4)

### (4) Introduction of RNP Complex

The procedure described in Example 1 (4) was followed.

### (5) Culturing of Dividing Cells and DNA Sampling

The RNP-transfected calluses cultured in (4) were incubated at 25° C. for 48 hours and then sampled. The extracted DNA sample was used as a template for PCR amplification of the target site. PCR was performed on the extracted DNA sample using primers (SEQ ID NO: 5 and 6). Other operations were performed in accordance with the procedure described in Example 2 (5).

*Primer sequences
OsLCYe5_Fw:
(SEQ ID NO: 5)
AGGGAAGGAGCAGGAGGGTTGTG
OsLCYe5_Rv:
(SEQ ID NO: 6)
GGAGTAGTGATATGATTTATTTACTGCTAC

### (6) Evaluation of Genome Editing Efficiency

DNA was extracted, purified, and sequenced without restriction enzyme cleavage. Those in which a mutation was introduced into the target gene sequence were determined as target-gene mutant cells. As a result, it was confirmed that the mutation was introduced into the OsLCYε target gene in the cells.

TABLE 2
Genome editing of the OsLCYε5 gene

| RNP | Culture | Culture | Editing |
|---|---|---|---|
| concentration | time | temperature | rate |
| (pmol) | (hours) | (° C.) | (%) |
| 100 | 48 | 25 | 0.007 |
| 200 | 48 | 25 | 0.015 |
| 600 | 48 | 25 | 0.018 |
| 100 | 48 | 35 | 0.016 |

### [Example 4] Production of Plants with Genome-Edited OsPDS Gene

### (1) Preparation of Test Plant Cells

The procedure described in Example 1 (1) was followed.

### (2) Preparation of an Acicular Inorganic Compound

The procedure described in Example 1 (2) was followed.

### (3) Preparation of a RNP Complex

The procedure described in Example 1 (3) was followed.

### (4) Preparation of a Plasmid Carrying a Resistance Gene

pCH, which carries an expression cassette of hygromycin resistance gene (hygromycin phosphotransferase gene), was used as a plasmid carrying a resistance gene. The plasmid (pCH) was dissolved in TE buffer (Tris-HCl 10 mM, EDTA 1 mM, pH 8.0) at a concentration of 1 mg/mL, and 10 μL of R2D2 medium was added to 20 μL of the resulting pCH solution (containing 20 μg), and mixed. The resulting solution was then added to the tube containing the mixture of the calluses, the whisker, and the RNP described above, and shaken thoroughly to obtain the mixture.

### (5) Introduction of RNP Complex

The procedure described in Example 1 (4) was followed.

### (6) Culturing of Dividing Cells

The RNP-transfected calluses were placed in a 3.5-cm petri dish, to which 3 mL of R2D2 medium was added, and the calluses were cultured at 28° C. in the light (2,000 lux, 16 hours illumination per day) using a rotary shaker (50 rpm/min) to thereby obtain dividing cells.

The cells were cultured according to the procedure described in (5) above, and on the third day of culture (72 hours), 3 mL of the suspension of these dividing cells was spread evenly on solid medium, which were prepared by solidifying 30 mL of N6 medium (pH 5.8) containing 2 mg/L of 2,4-D, 30 g/L of sucrose, 3 g/L of gelite, and 50 mg/L of hygromycin in a petri dish of 9 cm diameter. The liquid medium of the suspension was aspirated with a pipette and discarded. The cells were then incubated at 28° C. in the light (2,000 lux, 16 hours illumination per day) for 20 days to obtain hygromycin-resistant cells.

In the said resistant cells, whitened calluses were obtained due to mutation of the rice phytoene desaturase (PDS) gene. Thus, it was confirmed that genome editing of the PDS gene had occurred. The editing efficiency of the genome-edited mutant cells in the RNP-transfected group relative to the original DNA sequence was calculated.

TABLE 3
Genome editing of the OsPDS gene

| | | | Number of | | |
|---|---|---|---|---|---|
| | Number | RNP | hygromycin | Number of | Editing |
| Test | of treated | concentration | resistant | white | rate |
| No. | calluses | (pmol) | calluses | calluses | (%) |
| 1 | 4,500 | 100 | 22 | 15 | 0.33 |
| | 4,500 | 20 | 27 | 9 | 0.20 |
| 2 | 1,500 | 100 | 10 | 7 | 0.47 |
| | 1,500 | 40 | 3 | 1 | 0.07 |

Editing rate: Percentage of white calluses to treated calluses

(7) Regeneration of Plants from Genome-Edited Cells

Hygromycin-resistant and whitened cultured cells (1 cm in diameter) obtained in (6) above were placed in MS medium (pH 5.8) with a ½ concentration of inorganic salts containing 30 g/L sucrose, 30 g/L sorbitol, 0.3 mg/L benzyladenine, 0.3 mg/L naphthyl acetic acid, and 3 g/L gelurite, such that 6 calluses were placed per 9-cm-diameter petri dish. After 50 days of incubation at 28° C. in the light (2,000 lux, 16 hours of illumination per day), buds regenerated from the cultured cells.

The regenerated buds (grown to 3 to 5 mm in length) were transplanted one by one into solid medium prepared by solidifying 30 mL of MS medium with a ½ concentration of inorganic salts containing 30 g/L sucrose and 3 g/L gelrite in a test tube (15 cm long×4 cm diameter), and cultured for 30 days, resulting in plants with roots formed at the base of the growing shoots. A photograph of one of the resulting rice plants with its OsPDS gene genome-edited is shown in FIG. 2.

### (8) Confirmation of Genome Editing by Genome Analysis of Regenerated Plants

To analyze mutations caused by genome editing, a region of approximately 400 bp around the target site of the gRNA was amplified by short DNA region PCR and subjected to next-generation sequencing analysis. 50 mg of leaves of a regenerated plant were placed in a 1.5-mL volume microtube, into which 300 μL of 20 mM Tris-HCl buffer (pH 7.5) containing 10 mM EDTA was added. The mixture was ground, combined with 20 mL of 20% SDS, and heated at 65° C. for 10 minutes. The resulting mixture was combined with 100 μL of 5 M potassium acetate, placed in ice for 20 minutes, centrifuged at a centrifugal acceleration of 1,700×g for 20 minutes. 200 μL of isopropanol was added to the resulting supernatant, inverted and stirred, centrifuged again at a centrifugal acceleration of 1,700× g for 20 minutes, dried under reduced pressure, and dissolved in 100 μL of TE buffer to obtain DNA.

SEQ ID NO:2 and 3 were used as PCR primers. PCR was performed on the extracted DNA sample using the primers. KOD one was used as the PCR enzyme. An amplification cycle of 98° C.: 10 seconds, 55° C.: seconds, and 68° C.: 1 second was repeated for 35 cycles. The amplified products were used as samples for DNA sequencing analysis by next-generation sequencer (NGS). The PCR product from each amplified treatment was subjected to NGS analysis by Miseq (Illumina) to confirm that DNA editing had occurred.

### [Example 5] Production of Plants with Genome-Edited β-Carotene Gene

### (1) Preparation of Test Plant Cells

The procedure described in Example 1 (1) was followed.

### (2) Preparation of an Acicular Inorganic Compound

The procedure described in Example 1 (2) was followed.

### (3) Preparation of a RNP Complex

The procedure described in Example 2 (3) was followed.

### (4) Preparation of a Plasmid Carrying a Resistance Gene

The procedure described in Example 3 (4) was followed.

### (5) Introduction of RNP Complex

The target sequence is indicated in SEQ ID NO:7. The procedure in Example 3 (5) was followed except that the concentration of the RNP solution was adjusted to 100 μmol. *The target sequence of the OsLCYβ gene (sgRNA sequence)

OsLCYb2:
(SEQ ID NO: 7)
CTCCGTCTGCGCCATCGACC

### (6) Culturing of Dividing Cells

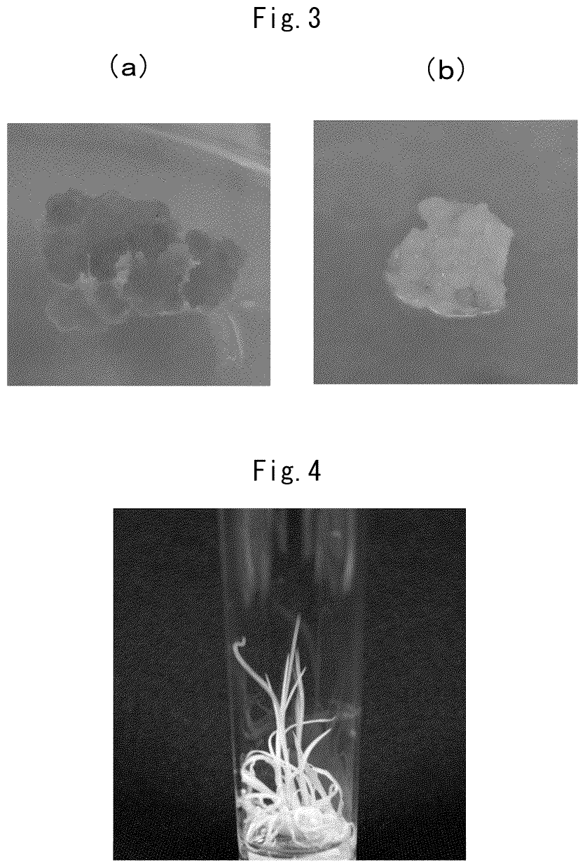

The procedure described in Example 3 (6) was followed. Among the resistant cells, reddish calluses were obtained due to mutation of the rice OsLCYβ gene, confirming that genome editing of the LCYβ gene occurred and β-carotene was accumulated. A photograph of a rice callus with genome-edited OsLCYβ gene is shown in shown in FIG. 3(a), and a photograph of a non-genome-edited rice callus in FIG. 3(b).

(7) Regeneration of Plants from Genome-Edited Cells

The procedure described in Example 3 (7) was followed. A photograph of a resulting rice plant with genome-edited OsLCYβ gene is shown in FIG. 4.

### (8) Confirmation of Genome Editing by Genome Analysis of Regenerated Plants

To analyze mutations caused by genome editing, a region of approximately 400 bp around the target site of the gRNA was amplified by short DNA region PCR and subjected to next-generation sequencing analysis. SEQ ID NO:8 and 9 were used as PCR primers.

*LCYb2_Fw:
(SEQ ID NO: 8)
TGCTCTCCCTCGACCTCC
*LCYb2_Rv:
(SEQ ID NO: 9)
TTGTGGAACGTGACGCCAT

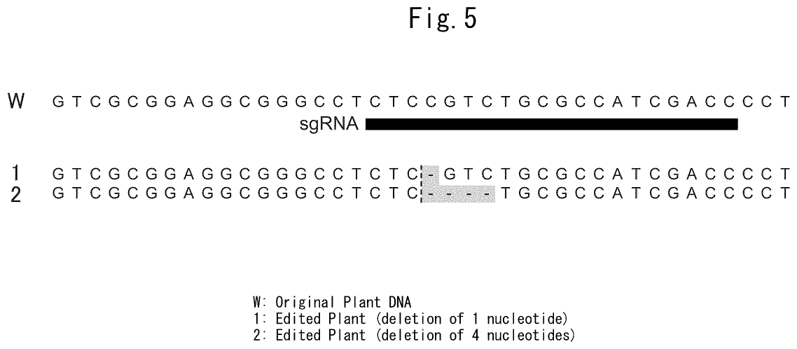

The PCR product from each treatment was subjected to NGS analysis by Miseq (Illumina). The obtained DNA sequence of the OsLCYβ gene is shown in FIG. 5. In the re-differentiated plants obtained from red callus, deletions of one and four nucleotides were found to be introduced.

### INDUSTRIAL APPLICABILITY

The present invention can be widely used in various industrial fields where plant genomic variants are involved, such as agriculture, pharmaceutical, and enzyme industries.

1. A method of editing the genome of a plant, comprising the steps of:
(i) mixing a plant cell or a tissue containing a plant cell with a site-specific DNA-modifying protein and an acicular inorganic compound in liquid medium and subjecting the mixture to disturbance to puncture the cell with the acicular inorganic compound and introduce the site-specific DNA-modifying protein into the cell; and
(ii) culturing the plant cell or the tissue containing the plant cell into which the site-specific DNA-modifying protein has been introduced at step (i) to generate a specific DNA mutation at a target site in the genome of the plant cell.

2. The method according to claim 1, wherein a guide RNA is co-mixed in the mixing at step (i).

3. A method of editing the genome of a plant, comprising the steps of:
(i) mixing a plant cell or a tissue containing a plant cell that constitutively or inductively expresses a site-specific DNA-modifying protein with a guide RNA and an acicular inorganic compound in liquid medium and subjecting the mixture to disturbance to puncture the plant cell with the acicular inorganic compound and cell and introduce the guide RNA into the cell, and
(ii) culturing the plant cell or the tissue containing the plant cell into which the guide RNA has been introduced to generate a specific DNA mutation at a target site in the genome of the plant cell.

4. The method according to claim 2, wherein the site-specific DNA-modifying protein is selected from the group consisting of Cas protein, zinc finger nuclease (ZFN), and TAL effector nuclease (TALEN).

5. The method according to claim 4, wherein the site-specific DNA-modifying protein forms a ribonucleoprotein (RNP) complex with the guide RNA.

6. The method according to claim 1, wherein the concentration of the ribonucleoprotein complex is from 20 to 800 μmol.

7. The method according to claim 1, wherein the maximum diameter of the plant cell or the tissue-contained plant cell is 1 mm or less.

8. The method according to claim 1, wherein the disturbance at step (i) is carried out by centrifugation and/or ultrasonication.

9. The method according to claim 8, wherein the ultrasonication at step (i) is carried out by applying ultrasonic waves with a frequency of from 10 to 60 kHz and an intensity of from 0.1 to 1 W/cm² for 30 seconds to 2 minutes.

10. The method according to claim 1, wherein a plasmid carrying a selection marker gene is co-mixed in the mixing at step (i).

11. The method according to claim 1, wherein the culturing at step (ii) is carried out at a temperature of from 25 to 40° C. for 1 to 72 hours.

12. A method for producing a genome-edited plant, comprising the step of editing the genome of a plant cell step using the method according to claim 2.

13. A method for producing a genome-edited plant seed, comprising the step of editing the genome of a plant cell step using the method according to claim 2.

14-15. (canceled)

16. The method according to claim 3, wherein the site-specific DNA-modifying protein is selected from the group consisting of Cas protein, zinc finger nuclease (ZFN), and TAL effector nuclease (TALEN).

17. The method according to claim 16, wherein the site-specific DNA-modifying protein forms a ribonucleoprotein (RNP) complex with the guide RNA.

18. The method according to claim 3, wherein the concentration of the ribonucleoprotein complex is from 20 to 800 μmol.

19. The method according to claim 3, wherein the maximum diameter of the plant cell or the tissue-contained plant cell is 1 mm or less.

20. The method according to claim 3, wherein the disturbance at step (i) is carried out by centrifugation and/or ultrasonication.

21. The method according to claim 20, wherein the ultrasonication at step (i) is carried out by applying ultrasonic waves with a frequency of from 10 to 60 kHz and an intensity of from 0.1 to 1 W/cm² for 30 seconds to 2 minutes.

22. The method according to claim 3, wherein a plasmid carrying a selection marker gene is co-mixed in the mixing at step (i).

23. The method according to claim 3, wherein the culturing at step (ii) is carried out at a temperature of from 25 to 40° C. for 1 to 72 hours.

24. A method for producing a genome-edited plant, comprising the step of editing the genome of a plant cell step using the method according to claim 3.

25. A method for producing a genome-edited plant seed, comprising the step of editing the genome of a plant cell step using the method according to claim 3.
